(19) United States
(12) Patent Application Publication
(10) Pub. No.: US 2011/0268779 A1
(43) Pub. Date: Nov. 3, 2011

(54) GUM COMPOSITIONS

(76) Inventor: Leigh Trevor Canham, Worcestershire (GB)

(21) Appl. No.: 13/130,138

(22) PCT Filed: Nov. 19, 2009
(86) PCT No.: PCT/GB2009/051572

§ 371 Date: Jul. 18, 2011

(30) Foreign Application Priority Data
Nov. 19, 2008 (GB) 0821186.4

(51) Int. Cl. A61K 9/68; C01B 33/113; A61K 8/02; A61P 1/02; A61P 3/04; A61P 25/30; C01B 33/02; A61Q 11/00; B82Y 30/00

(52) U.S. Cl. 424/401; 423/348; 423/325; 424/48; 977/773

(57) ABSTRACT

A chewing gum composition comprising porous silicon is described.

### FIELD OF THE INVENTION

This invention relates to chewing gum compositions comprising porous silicon and methods of making said compositions. This invention also relates to various uses of said chewing gum compositions.

### BACKGROUND OF THE INVENTION

There are numerous challenges facing the manufacturers of chewing gum. For example, it is notoriously difficult to bind key active ingredients, such as flavours, with chewing gum and hence deliver said actives effectively. Typically, only about 40 wt % of an active based on the total weight of the gum is released into the mouth during chewing (mastication) meaning that very few gums provide intense flavours that persist for many minutes. Rapid loss of flavour remains the most challenging concern for gum manufacturers. Other challenges facing manufacturers include achieving tasty sugar-free gums due to the incompatibility of sweeteners with other components. Incorporating vitamins and other nutrients also presents particular challenges as does the prevention or minimisation of the gum significantly changing colour (e.g. improving fade resistance) during storage when it may be exposed to light and/or heat.

There is also an increasing interest in so-called functional gums which possess specific actives which perform functions such as tooth cleaning or whitening and breath freshening. There is also growing interest in so-called nutraceutical gums which tend to be viewed more as dietary supplements possessing a range of incorporated nutrients or drugs for specific inventions.

There is a continued need for alternative and preferably improved formulations for effectively delivering specific ingredients to the human or animal teeth and/or other oral surfaces. The present invention seeks to address some of the issues and problems set out above and is partly based on the surprising finding that porous silicon may be used in chewing gum formulations to; effectively deliver at least one active agent to the human or animal teeth and/or other oral surfaces, (other oral surfaces include one or more of the tongue, cheeks, gums, throat), retain volatile flavours during storage and triggered release thereof, provide acceptable or improved fade resistance.

### SUMMARY OF THE INVENTION

According to a first aspect of the present invention, a chewing gum composition comprising porous silicon is provided.

According to a second aspect of the present invention, there is provided a production process for said chewing gum composition according to the first aspect of the present invention, comprising blending said porous silicon and other components of the chewing gum composition.

According to a third aspect of the invention, the use of porous silicon in a chewing gum composition is provided.

According to a further aspect of the present invention a method of treating and/or cleaning the teeth of a human or animal comprising chewing a chewing gum composition according to the first aspect of the present invention is provided. The method may be a cosmetic method.

According to a further aspect of the present invention, a method for preventing and/or reducing stain and/or plaque and/or gingivitis and/or calculus comprising chewing the chewing gum composition according to the first aspect of the present invention is provided.

According to a further aspect of the present invention, a chewing gum composition according to the first aspect of the invention for use in the treatment and/or prevention of plaque and/or gingivitis and/or calculus is provided.

According to a further aspect of the present invention, a cosmetic method for reducing stain comprising chewing the chewing gum composition according to the first aspect of the present invention is provided.

According to a further aspect of the present invention, the use of porous silicon for maintaining the colour of a chewing gum composition during storage is provided.

The porous silicon may comprise at least one ingredient for delivery to human and/or animal teeth and/or other oral surfaces. Suitable ingredients include actives such as one or more of: sweetener, flavour, breath freshening agent, vitamin, antimicrobial, antibacterial, remineralizing agent, anti-plaque agent, anti-gingivitis agent, anti-calculus agent, tooth whitening agent, herbal extract, pain-relief agent, sensate, cooling agent, warming agent, colouring agent (e.g. one or more pigments), stimulant, essential oil, slimming agent, cholesterol lowering agent, anti-smoking agent, nutrient. The porous silicon may be loaded with the at least one ingredient which may be entrapped in the silicon pores.

The use of porous silicon in chewing gum compositions according to the present invention seeks to provide one or more of the following: improved bioavailability, sustained flavour release in the mouth, targeted delivery for use in connection with mouth ulcers and/or toothache and/or sore throat, enteric release in the intestine, sequential burst release in the mouth, taste masking, acceptable or improved buccal absorption, protection of the at least one active ingredient during the manufacturing process, acceptable or improved colour retention of the gum composition during storage.

### DETAILED DESCRIPTION OF THE INVENTION

### Porous Silicon

As used herein, and unless otherwise stated, the term “silicon” refers to solid elemental silicon. For the avoidance of doubt, and unless otherwise stated, it does not include silicon-containing chemical compounds such as silica, silicates or silicones, although it may be used in combination with these materials.

The physical forms of porous silicon which are suitable for use in the present invention may be chosen from or comprise amorphous silicon, single crystal silicon and polycrystalline silicon (including nanocrystalline silicon, the grain size of which is typically taken to be 1 to 100 nm) and including combinations thereof. The silicon may be surface porosified, for example, using a stain etch method or more substantially porosified, for example, using an anodisation technique. Following porosification some non-porosified silicon, such as bulk silicon, may be present with the porous silicon. The porous silicon is advantageously selected from microporous and/or mesoporous silicon. Mesoporous silicon contains pores having a diameter in the range of 2 to 50 nm. Microporous silicon contains pores possessing a diameter less than 2 nm.

The average pore diameter is measured using a known technique. Mesopore diameters are measured by very high resolution electron microscopy. This technique and other suitable techniques which include gas-adsorption-desorption analysis, small angle x-ray scattering, NMR spectroscopy or thermoporometry, are described by R. Herino in “Properties of Porous Silicon”, chapter 2.2, 1997. Micropore diameters are measured by xenon porosimetry, where the Xe¹²⁹ nmr signal depends on pore diameter in the sub 2 nm range.

The porous silicon may have a BET surface area of 10 m²/g to 800 m²/g for example 100 m²/g to 400 m²/g. The BET surface area is determined by a BET nitrogen adsorption method as described in Brunauer et al., J. Am. Chem. Soc., 60, p 309, 1938. The BET measurement is performed using an Accelerated Surface Area and Porosimetry Analyser (ASAP 2400) available from Micromeritics Instrument Corporation, Norcross, Georgia 30093. The sample is outgassed under vacuum at 350° C. for a minimum of 2 hours before measurement.

Generally, the degree of porosity may be up to about 90 vol %. For the delivery of one or more active materials, the porosity of the silicon may be about 20 to 90 vol %, for example, 30 to 80 vol %. Non-porous silicon may be included in the gum compositions according to the present invention in combination with the porous silicon. As such, the porosity of the silicon may be 0 to 90 vol %, for example 0 to 80 vol %. It is also possible to blend proportions of porous silicon which possess different ranges of porosity.

The purity of the porous silicon may be about 95 to 99.99999% pure, for example about 95 to 99.99% pure. So-called metallurgical silicon which may also be used in the chewing gum compositions has a purity of about 98 to 99.5%.

The porous silicon may consist of, consist essentially of, or comprise resorbable silicon. The porous silicon may consist of, consist essentially of, or comprise bioactive silicon.

Bioactive materials are highly compatible with living tissue and capable of forming a bond with tissue by eliciting a specific biological response. Bioactive materials may also be referred to as surface reactive biomaterials. Bioactive silicon comprises a nanostructure and such nanostructures include: (i) microporous silicon, mesoporous silicon either of which may be single crystal silicon, polycrystalline silicon or amorphous silicon; (ii) polycrystalline silicon with nanometre size grains; (iii) nanoparticles of silicon which may be amorphous or crystalline.

Though not wishing to be bound by a particular theory, it is believed that the use of bioactive porous silicon, according to the present invention, generates silicic acid in-situ which promotes remineralisation of the tooth. The porous silicon, which may be bioactive silicon, may comprise additional components such as a source of calcium and/or phosphate and/or fluoride in order to aid, for example, in the remineralisation process. This includes the remineralisation of subsurface dental enamel and/or mineralising tubules in dentin thereby counteracting caries and/or hypersensitivity. At least about 10 ppm of calcium ions may be present, with the upper limit being about 35,000 ppm. The concentration of phosphate ions may typically be in the range of about 250 to 40,000 ppm.

In order to deliver significant flavour over a significant amount of time, one or more of a number of parameters may be varied. These include the particle size distribution, including the mean particle size (d₅₀), yield strength, porosity. For example, the mean particle size may be about 100 nm to 100 μm, for example about 1 to 20 μm. For example, the yield strength of porous silicon microparticles is about 1 to 7000 MPa, for example, about 10 to 1000 MPa.

The use of porous silicon according to the present invention may impart a visually appealing appearance to the teeth and, as such, according to a further aspect of the present invention, the use of porous silicon in a chewing gum composition for modifying the appearance of teeth is provided. This may include a glittering or glinting appearance. By using mirrors, which reflect different wavelengths of light, specific colouration of teeth may be effected. This may be achieved by varying the porosities of adjacent layers comprising porous silicon between low and high porosity layers. Typically, the low porosity layers may have a porosity of up to about 65 vol %, for example about 25 vol % to 65 vol % and the high porosity layers may have a porosity of at least about 60 vol %, for example about 60 vol % to 95 vol %. Each mirror may comprise greater than 10 layers or greater than 100 layers, or greater than 200 layers or greater than or equal to 400 layers. Each layer from which the mirrors are formed has a different refractive index to its neighbouring layer or layers such that the combined layers form a Bragg stack mirror. Specific colours may also be imparted to silicon particles by surface porosification using stain etching or partial oxidation. In general, porous silicon possessing a particle size less than 10 μm may be used in connection with the present invention in providing optical effects.

The total amount of porous silicon present in the chewing gum composition may be about 0.001 wt % to 10 wt % based on the total weight of the chewing gum composition and the unloaded weight of the silicon, for example 0.1 wt % to 10 wt %.

### Silicon Manufacture and Processing

Methods for making various forms of silicon which are suitable for use in the present invention are described below. The methods described are well known in the art.

In PCT/GB96/01863, the contents of which are incorporated herein by reference in their entirety, it is described how bulk crystalline silicon can be rendered porous by partial electrochemical dissolution in hydrofluoric acid based solutions. This etching process generates a silicon structure that retains the crystallinity and the crystallographic orientation of the original bulk material. Hence, the porous silicon formed is a form of crystalline silicon. Broadly, the method involves anodising, for example, a heavily boron doped CZ silicon wafer in an electrochemical cell which contains an electrolyte comprising a 20% solution of hydrofluoric acid in an alcohol such as ethanol, methanol or isopropylalcohol (IPA). Following the passing of an anodisation current with a density of about 50 mAcm⁻², a porous silicon layer is produced which may be separated from the wafer by increasing the current density for a short period of time. The effect of this is to dissolve the silicon at the interface between the porous and bulk crystalline regions. Porous silicon may also be made using the so-called stain-etching technique which is another conventional method for making porous silicon. This method involves the immersion of a silicon sample in a hydrofluoric acid solution containing a strong oxidising agent. No electrical contact is made with the silicon, and no potential is applied. The hydrofluoric acid etches the surface of the silicon to create pores.

Mesoporous silicon may be generated from a variety of non-porous silicon powders by so-called “electroless electrochemical etching techniques”, as reviewed by K. Kolasinski in Current Opinions in Solid State & Materials Science 9, 73 (2005). These techniques include “stain-etching”, “galvanic etching”, “hydrothermal etching” and “chemical vapour etching” techniques. Stain etching results from a solution containing fluoride and an oxidant. In galvanic or metal-assisted etching, metal particles such as platinum are also involved. In hydrothermal etching, the temperature and pressure of the etching solution are raised in closed vessels. In chemical vapour etching, the vapour of such solutions, rather than the solution itself is in contact with the silicon. Mesoporous silicon can be made by techniques that do not involve etching with hydrofluoric acid. An example of such a technique is chemical reduction of various forms of porous silica as described by Z. Bao et al in Nature vol. 446 8th March 2007 p 172-175 and by E. Richman et al. in Nano Letters vol. 8(9) p 3075-3079 (2008). If this reduction process does not proceed to completion then the mesoporous silicon contains varying residual amounts of silica.

Following its formation, the porous silicon may be dried. For example, it may be supercritically dried as described by Canham in Nature, vol. 368, (1994), pp 133-135. Alternatively, the porous silicon may be freeze dried or air dried using liquids of lower surface tension than water, such as ethanol or pentane, as described by Bellet and Canham in Adv. Mater, 10, pp 487-490, 1998.

Silicon hydride surfaces may, for example, be generated by stain etch or anodisation methods using hydrofluoric acid based solutions. When the silicon, prepared, for example, by electrochemical etching in HF based solutions, comprises porous silicon, the surface of the porous silicon may or may not be suitably modified in order, for example, to improve the stability of the porous silicon in the chewing gum composition. In particular, the surface of the porous silicon may be modified to render the silicon more stable in alkaline conditions. The surface of the porous silicon may include the external and/or internal surfaces formed by the pores of the porous silicon.

In certain circumstances, the stain etching technique may result in partial oxidation of the porous silicon surface. The surfaces of the porous silicon may therefore be modified to provide: silicon hydride surfaces; silicon oxide surfaces wherein the porous silicon may typically be described as being partially oxidised; or derivatised surfaces which may possess Si—O—C bonds and/or Si—C bonds. Silicon hydride surfaces may be produced by exposing the porous silicon to HF.

Silicon oxide surfaces may be produced by subjecting the silicon to chemical oxidation, photochemical oxidation or thermal oxidation, as described for example in Chapter 5.3 of Properties of Porous Silicon (edited by L. T. Canham, IEE 1997). PCT/GB02/03731, the entire contents of which are incorporated herein by reference, describes how porous silicon may be partially oxidised in such a manner that the sample of porous silicon retains some elemental silicon. For example, PCT/GB02/03731 describes how, following anodisation in 20% ethanoic HF, the anodised sample was partially oxidised by thermal treatment in air at 500° C. to yield a partially oxidised porous silicon sample.

Following partial oxidation, an amount of elemental silicon will remain. The silicon particles may possess an oxide content corresponding to between about one monolayer of oxygen and a total oxide thickness of less than or equal to about 4.5 nm covering the entire silicon skeleton. The porous silicon may have an oxygen to silicon atomic ratio between about 0.04 and 2.0, and preferably between 0.60 and 1.5. Advantageously, the ratio may be 1.5 to 1.99, e.g. 1.8 to 1.99, particularly in connection with pigment protection and maintaining colour and preferably when the porous silicon is mesoporous silicon. Oxidation may occur in the pores and/or on the external surface of the silicon.

Derivatised porous silicon is porous silicon possessing a covalently bound monolayer on at least part of its surface. The monolayer typically comprises one or more organic groups that are bonded by hydrosilylation to at least part of the surface of the porous silicon. Derivatised porous silicon is described in PCT/GB00/01450, the contents of which are incorporated herein by reference in their entirety. PCT/GB00/01450 describes derivatisation of the surface of silicon using methods such as hydrosilyation in the presence of a Lewis acid. In that case, the derivatisation is effected in order to block oxidation of the silicon atoms at the surface and so stabilise the silicon. Methods of preparing derivatised porous silicon are known to the skilled person and are described, for example, by J. H. Song and M. J. Sailor in Inorg. Chem. 1999, vol 21, No. 1-3, pp 69-84 (Chemical Modification of Crystalline Porous Silicon Surfaces). Derivitisation of the silicon may be desirable when it is required to increase the hydrophobicity of the silicon, thereby decreasing its wettability. Preferred derivatised surfaces are modified with one or more alkyne groups. Alkyne derivatised silicon may be derived from treatment with acetylene gas, for example, as described in “Studies of thermally carbonized porous silicon surfaces” by J. Salonen et al in Phys Stat. Solidi (a), 182, pp 123-126, (2000) and “Stabilisation of porous silicon surface by low temperature photoassisted reaction with acetylene”, by S. T. Lakshmikumar et al in Curr. Appl. Phys. 3, pp 185-189 (2003). Mesoporous silicon may be derivatised during its formation in HF-based electrolytes, using the techniques described by G. Mattei and V. Valentini in Journal American Chemical Society vol 125, p 9608 (2003) and Valentini et al., Physica Status Solidi (c) 4 (6) p2044-2048 (2007).

The surface chemistry of the porous silicon may be adapted depending on the particular application. For example, the surface chemistry may be tailored in order to promote binding to teeth and/or gums and/or the tongue and/or cheeks, and/or throat.

The porous silicon may also comprise a capping layer in order to prevent release of the loaded ingredient prior to application to the human or animal or too soon following application. In particular, the porous silicon may be capped using ultrathin capping layers or beads around the loaded porous silicon. The capping layers may assist in providing retention of the loaded ingredient over a number of months of storage, for example from about 1 year up to about 5 years. The capping layer may also be designed to trigger active release of the loaded ingredient through site-specific degradation when in contact with the human or animal teeth and/or other oral surfaces. Suitable capping materials include one or more of the following: gum polymer, whitening agent, metal salt, filler, sweetening agent, wax, thickener, colouring agent, fat, oil, polyol. In particular, suitable capping materials include silicon, silicon dioxide, shellac, calcium phosphates, calcium sulphate, calcium carbonate, titanium dioxide, magnesium carbonate, gum Arabic, cellulose, polydextrose, sorbitol, xylitol, mannitol, polyvinylpyrrolidone (PVP), maltodextrin and blends thereof, for example gum Arabic and maltodextrin. Suitable methods for capping the porous silicon include spray drying, fluidized bed coating, pan coating, modified microemulsion techniques, melt extrusion, spray chilling, complex coacervation, vapour deposition, solution precipitation, emulsification, supercritical fluid techniques, physical sputtering, laser ablation, and thermal evaporation. The capping layer may, for example, be degraded by contact with saliva, or in the case wherein the loaded ingredient is intended for enteric delivery when in contact with intestinal fluids. The capping layer may remain essentially intact during the chewing process and the at least one ingredient for delivery to the human or animal may be released by fracture of at least some of the porous silicon particles.

### Particulate Silicon

The silicon is typically present in particulate form. Methods for making silicon powders such as silicon microparticles and silicon nanoparticles are well known in the art. Silicon microparticles are generally taken to mean particles of about 1 to 1000 μm in diameter and silicon nanoparticles are generally taken to mean particles possessing a diameter of about 100 nm and less. Silicon nanoparticles therefore typically possess a diameter in the range of about 1 nm to about 100 nm, for example about 5 nm to about 100 nm. Fully biodegradable mesoporous silicon typically has an interconnected silicon skeleton with widths in the 2-5 nm range. In connection with the present invention, mesoporous silicon particles possessing a diameter of 50 nm to 100 μm, for example, particularly 100 nm to 10 μm may be employed. Mesoporous silicon particles may also be produced by agglomerating small non-porous silicon particles into microparticles. The non-porous particles may possess a diameter of 50 to 1000 nm. Methods for making silicon powders are often referred to as “bottom-up” methods, which include, for example, chemical synthesis or gas phase synthesis. Alternatively, so-called “top-down” methods refer to such known methods as electrochemical etching or comminution (e.g. milling as described in Kerkar et al. J. Am. Ceram. Soc., vol. 73, pages 2879-2885, 1990.). PCT/GB02/03493 and PCT/GB01/03633, the contents of which are incorporated herein by reference in their entirety, describe methods for making particles of silicon, said methods being suitable for making silicon for use in the present invention. Such methods include subjecting silicon to centrifuge methods, or grinding methods. Porous silicon powders may be ground between wafers or blocks of crystalline silicon. Since porous silicon has lower hardness than bulk crystalline silicon, and crystalline silicon wafers have ultrapure, ultrasmooth surfaces, a silicon wafer/porous silicon powder/silicon wafer sandwich is a convenient means of achieving for instance, a 1-10 μm particle size from much larger porous silicon particles derived, for example, via anodisation.

The surface of silicon particles prepared by “top down” or “bottom up” methods may also be a hydride surface, a partially oxidised surface, a fully oxidised surface or a derivatised surface. Milling in an oxidising medium such as water or air will result in silicon oxide surfaces. Milling in an organic medium may result in, at least partial derivatisation of the surface. Gas phase synthesis, such as from the decomposition of silane, will result in hydride surfaces. The surface may or may not be suitably modified in order, for example, to improve the stability of the particulate silicon in the chewing gum composition.

Other examples of methods suitable for making silicon nanoparticles include evaporation and condensation in a subatmospheric inert-gas environment. Various aerosol processing techniques have been reported to improve the production yield of nanoparticles. These include synthesis by the following techniques: combustion flame; plasma; laser ablation; chemical vapour condensation; spray pyrolysis; electrospray and plasma spray. Because the throughput for these techniques currently tends to be low, preferred nanoparticle synthesis techniques include: high energy ball milling; gas phase synthesis; plasma synthesis; chemical synthesis; sonochemical synthesis.

Some methods of producing silicon nanoparticles are described in more detail below.

### High-Energy Ball Milling

High energy ball milling, which is a common top-down approach for nanoparticle synthesis, has been used for the generation of magnetic, catalytic, and structural nanoparticles, see Huang, “Deformation-induced amorphization in ball-milled silicon”, Phil. Mag. Lett., 1999, 79, pp 305-314. The technique, which is a commercial technology, has traditionally been considered problematic because of contamination problems from ball-milling processes. However, the availability of tungsten carbide components and the use of inert atmosphere and/or high vacuum processes has reduced impurities to acceptable levels. Particle sizes in the range of about 0.1 to 1 μm are most commonly produced by ball-milling techniques, though it is known to produce particle sizes of about 0.01 μm.

Ball milling can be carried out in either “dry” conditions or in the presence of a liquid, i.e. “wet” conditions. For wet conditions, typical solvents include water or alcohol based solvents.

### Gas Phase Synthesis

Silane decomposition provides a very high throughput commercial process for producing polycrystalline silicon granules. Although the electronic grade feedstock (currently about $30/kg) is expensive, so called “fines” (microparticles and nanoparticles) are a suitable waste product for use in the present invention. Fine silicon powders are commercially available. For example, NanoSi™ Polysilicon is commercially available from Advanced Silicon Materials LLC and is a fine silicon powder prepared by decomposition of silane in a hydrogen atmosphere. The particle size is 5 to 500 nm and the BET surface area is about 25 m²/g. This type of silicon has a tendency to agglomerate, reportedly due to hydrogen bonding and Van der Waals forces.

### Plasma Synthesis

Plasma synthesis is described by Tanaka in “Production of ultrafine silicon powder by the arc plasma method”, J. Mat. Sci., 1987, 22, pp 2192-2198. High temperature synthesis of a range of metal nanoparticles with good throughput may be achieved using this method. Silicon nanoparticles (typically 10-100 nm diameter) have been generated in argon-hydrogen or argon-nitrogen gaseous environments using this method.

### Chemical Synthesis

Solution growth of ultra-small (<10 nm) silicon nanoparticles is described in US 20050000409, the contents of which are incorporated herein in their entirety. This technique involves the reduction of silicon tetrahalides such as silicon tetrachloride by reducing agents such as sodium napthalenide in an organic solvent. The reactions lead to a high yield at room temperature.

### Sonochemical Synthesis

In sonochemistry, an acoustic cavitation process can generate a transient localized hot zone with extremely high temperature gradient and pressure. Such sudden changes in temperature and pressure assist the destruction of the sonochemical precursor (e.g., organometallic solution) and the formation of nanoparticles. The technique is suitable for producing large volumes of material for industrial applications. Sonochemical methods for preparing silicon nanoparticles are described by Dhas in “Preparation of luminescent silicon nanoparticles: a novel sonochemical approach”, Chem. Mater., 10, 1998, pp 3278-3281.

### Mechanical Synthesis

Lam et al have fabricated silicon nanoparticles by ball milling graphite powder and silica powder, this process being described in J. Crystal Growth 220(4), p 466-470 (2000), which is herein incorporated by reference in its entirety. Arujo-Andrade et al have fabricated silicon nanoparticles by mechanical milling of silica powder and aluminium powder, this process being described in Scripta Materialia 49(8), p 773-778 (2003).

An alternative method for making porous silicon from nanoparticles includes exposing nanoparticulate elemental silicon to a pulsed high energy beam. The high energy beam may be a laser beam or an electron beam or an ion beam. Preferably, the high energy beam creates a condition wherein the elemental silicon is rapidly melted, foamed and condensed. Preferably, the high energy beam is a pulsed laser beam.

### Agglomerated Particles

Silicon microparticles or nanoparticles may be transformed into a porous agglomerated form, suitable for use in the present invention, by thermal processing, compression techniques or by the application of centrifugal forces. The agglomerated forms comprise a unitary body with mesopores and/or micropores.

PCT/GB2005/001910 the contents of which are incorporated herein in their entirety describes how particulate silicon, which may or may not be porous, may be consolidated to form a multiplicity of bonded silicon particles typically under the influence of pressure. The pressure may, for example be applied uniaxially or isostatically. Typical uniaxial pressures may be in the range of 10 MPa to 5000 MPa and the isostatic pressure may be in the range of 10 MPa to 5000 MPa.

The consolidation may be carried out such that the unitary body or silicon structure formed possesses a surface area greater than 100 cm²/g, for example, greater than 1 m²/g.

The consolidation of the silicon particulate product may result in a porous unitary body, the pores being formed from the spaces between the bonded silicon particles. However, the free silicon particles may themselves be porous prior to consolidation, for example by the use of stain etching or anodisation techniques.

The consolidated product or so-called unitary body may itself be further porosified by anodisation or stain etching and/or may be fragmented. Fragmentation techniques include mechanical crushing or the use of ultrasonics.

The formation of the unitary body may be carried out within a selected temperature range. Cold pressing means that the consolidation is carried out up to a temperature of about 50° C. and from as low as −50° C.

The surface area of a silicon unitary body formed by a cold pressing technique may be high, relative to that of a silicon unitary body formed by a hot pressing technique. This is because hot pressing can result in rearrangement of the surface silicon atoms, causing cavities and defects to be removed.

The consolidation process may comprise combining the particulate silicon prior, and/or during and/or after consolidation with the ingredient or ingredients to be loaded in such a manner that the ingredient is located in the pores between the bonded silicon particles.

In the present invention, particle size distribution measurements, including the mean particle size (d₅₀/μm) of the porous silicon particles are measured using a Malvern Particle Size Analyzer, Model Mastersizer, from Malvern Instruments. A helium-neon gas laser beam is projected through a transparent cell which contains the silicon particles suspended in an aqueous solution. Light rays which strike the particles are scattered through angles which are inversely proportional to the particle size. The photodetector array measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silicon.

### Ingredients

The porous silicon may be loaded with one or more active ingredients. These ingredients include one or more of the following: sweetener, flavour agent, breath freshening agent, vitamin, antimicrobial, remineralizing agent, anti-plaque agent, anti-gingivitis agent, anti-calculus agent, tooth whitening agent, herbal extract, pain-relief agent, sensate, cooling agent, warming agent, colouring agent (e.g. pigment), stimulant, essential oil. The porous silicon may be loaded with the ingredient which may be entrapped in the silicon pores.

Typically, the one or more active ingredients are present in the range, in relation to the loaded silicon, of 1 to 90 wt %, for example 30 to 60 wt %.

The ingredient to be loaded with the porous silicon may be dissolved or suspended in a suitable solvent, and porous silicon particles may be incubated in the resulting solution for a suitable period of time. Both aqueous and non-aqueous slips have been produced from ground silicon powder and the processing and properties of silicon suspensions have been studied and reported by Sacks in Ceram. Eng. Sci. Proc., 6, 1985, pp 1109-1123 and Kerkar in J. Am. Chem. Soc. 73, 1990, pp 2879-85. The wetting of solvent will result in the ingredient penetrating into the pores of the silicon by capillary action, and, following solvent removal, the ingredient will be present in the pores. Preferred solvents are water, ethanol, and isopropyl alcohol, GRAS solvents and volatile liquids amenable to freeze drying. Liquid ingredients, e.g. liquid pigments may be mixed with the porous silicon.

In general, if the ingredient to be loaded has a low melting point and a decomposition temperature significantly higher than that melting point, then an efficient way of loading the ingredient is to melt the ingredient.

Higher levels of loading, for example, at least about 30 wt % of the loaded ingredient based on the loaded weight of the silicon may be achieved by performing the impregnation at an elevated temperature. For example, loading may be carried out at a temperature which is at or above the melting point of the ingredient to be loaded. Quantification of gross loading may conveniently be achieved by a number of known analytical methods, including gravimetric, EDX (energy-dispersive analysis by x-rays), Fourier transform infra-red (FTIR), Raman spectroscopy, UV spectrophotometry, titrimetric analysis, HPLC or mass spectrometry. If required, quantification of the uniformity of loading may be achieved by techniques that are capable of spatial resolution such as cross-sectional EDX, Auger depth profiling, micro-Raman and micro-FTIR.

The loading levels can be determined by dividing the volume of the ingredient taken up during loading (equivalent to the mass of the ingredient taken up divided by its density) by the void volume of the porous silicon prior to loading multiplied by one hundred.

### Sweeteners

Suitable one or more sweeteners include bulk sweeteners and high intensity artificial sweeteners. Bulk sweeteners can typically constitute 30-60 wt % of the gum weight. Bulk sweeteners are sugar based or non-sugar based. Suitable sugar-based sweeteners include sucrose, dextrose, maltose, trehalose, fructose, galactose. Non-sugar based sweeteners include polyols such as sorbitol, mannitol, xylitol, isomalt, erythritol, lactitol, maltitol. High intensity sweetening agents can be used alone or in combination with bulk sweeteners, including those listed above. Suitable examples include sucralose, aspartame, acesulfame salts, alitame, neotame, cyclamic acid, thaumatin, monellin. These are typically present at 0.001-5 wM of the total gum weight.

### Flavours

Suitable one or more flavours include oils or extracts derived from natural, agricultural, plant or food sources including lemon oil, lime oil, oils derived from honey, cherry, menthol, eucalyptus, peppermint, spearmint, liquorice, ginger, cinnamon and orange extracts. Other oils derived from plants and fruits include mint oils, clove oil, oil of wintergreen, cinnamic aldehyde, anise. Although natural flavours are preferred, synthetic or artificial flavouring agents may also be used. Suitable examples include ethyl butyrate and gamma decalactone,

### Breath Fresheners

Suitable one or more breath fresheners include agents which are able to either reduce the concentration of mouth odour causing bacteria, or absorb, adsorb, bind or otherwise nullify the volatile species responsible for “bad breath”. Suitable examples include antimicrobial agents such as triclosan and/or chlorohexidine. Other suitable examples include zinc salts such as zinc lactate, zinc oxide, zinc acetate. Other suitable examples include natural extracts obtained from tea, coriander, magnolia bark, tea tree oil, thyme and honey suckle.

### Vitamins

Suitable examples of one or more vitamins include vitamin A, B1, B12, C, D, E, K.

### Antimicrobials

Suitable one or more antimicrobials include miconazole, nystatin, triclosan, essential oils such as methyl salicylate, menthol, eucaplytol, thymol.

### Remineralising Agents

Re-mineralization refers to the reversal of tooth enamel demineralization. Hence suitable one or more re-mineralizing agents are those that either assist in building up enamel and/or assist in inhibiting its demineralization. Suitable examples include pH adjusting agents such as sodium bicarbonate. Other examples include compounds that can provide fluoride, calcium or potassium ions such as calcium fluoride, sodium fluoride, dicalcium phosphate, and osteopontin which is a complex of calcium phosphate nanoclusters and casein phophoprotein.

### Herbal Extracts

Suitable herbal extracts include alfalfa, aloe vera, basil, bay, bergamot, borage, chamomile, chervil, chives, cinnamon, coriander, dandelion, echinacea, elderflower, evening primrose, feverfew, ginseng, kelp, lavender, lemon balm, lime blossom, marigold, marjoram, meadowsweet, mint, nasturtium, oregano, parsley, peppermint, rocket, rosehip, rosemary, safflower, sage, sorrel, thyme, valerian, watercress.

### Pain Relief Agents

Suitable one or more pain relief agents include chemical compounds used in conventional cough and cold remedies including topical anesthetics and throat soothing agents such as ethylaminobenzoate diperodon hydrochloride, benzocaine, bezyl alcohol.

### Cooling Agents

Several chemical compounds are known to induce a cooling sensation in the oral cavity, when brought into contact with the mucous membranes of the mouth and throat. A suitable example is the menthol of peppermint oil, used to create cooling sensations in toothpaste, chewing gum and mouthwash. Other examples of physiological cooling agents suitable for use in gum include p-menthane carboxamides (PMC), acrylic carboxamides (AC), menthyl lactate (ML), menthyl succinate (MS). Such active agents may be used in gum at levels of 0.001-2 wt % of the gum weight.

### Sensates

Suitable sensates include menthyl glutarate, isopulegol.

### Warming Agents

Several chemical compounds are known to provide a warming sensation in the oral cavity, and can enhance the perception of gum flavour, sweetness or other organoleptic attributes. Suitable examples of one or more warming agents include vanillyl alcohol n-butylether, vanillyl alcohol n-propylether, gingerol, paradol, capsaicin, capsicum, oleoresin.

### Anticaries Agent

Suitable anticaries agent, include a source of fluoride ions. The source of fluoride ions may be sufficient to supply about 25 ppm to 5000 ppm of fluoride ions, for example about 525 to 1450 ppm. Suitable examples of anticaries agents include one or more inorganic salts such as soluble alkali metal salts including sodium fluoride, potassium fluoride, ammonium fluorosilicate, sodium fluorosilicate, sodium monofluorophosphate, and tin fluorides such as stannous fluoride.

### Whitening Agents

These agents are able to modify the colour of teeth in order to make them appear whiter. They utilize either physical, for example, optical masking effects, or assist in the chemical bleaching of stains on or in tooth surfaces, for example, through oxidation processes. Suitable examples of one or more such whitening agents are listed in the CTFA Cosmetic Ingredient Handbook 3rd Edition (1982) from the Cosmetic and Fragrance Association, Washington DC, USA. Specific examples include talc, mica, calcium carbonate, icelandic moss, bamboo, silica, titanium dioxide, starch, iron oxides of various colours, nylon powders, polystyrene powders.

### Anti-calculus Agents

Calculus is the hardened deposit of mineralized plaque and saliva that can build up on teeth. Anti-calculus agents therefore help prevent or reduce the formation of such hardened deposits. Suitable examples of one or more anti-calculus agents include phosphate, pyrophosphate, polyacrylate, vitamin C, citric acid and acetic acid.

### Anti-gingivitis Agents

Gingivitis is the gum inflammation around teeth that is often caused by plaque build-up or food retention. Anti-gingivitis agents thus assist in preventing or treating such inflammation. Suitable one or more anti-gingivitis agents include anti-inflammatory agents such as aspirin, ibuprofen, piroxicam. Others include psycotherapeutic agents such as thorazine, lorazepam, sorentil. Other examples include chlorohexidine, sage extract, aloe vera extract and myrrh.

### Colouring Agents

Suitable colouring agents, e.g. pigments, include those already approved for use in food products like chewing gum, or oral hygiene products like toothpaste. These may be synthetic pigments known as FD & C dyes and lakes, as listed in the Kirk-Othmer Encyclopedia of Chemical Technology, 2nd Edition Vol. 5 p 857-884. Examples of pigments currently in use in chewing gum are allura red (E133), brilliant blue FCF (E133), carbon black (E153), caramel (E150d), yellow 5 lake and blue 1 lake. The pigments may be natural pigments already used in other foodstuffs, such as elderberry, grape, tomato, safflower and cocoa derived pigments.

More preferred pigments are the natural or nature-identical pigments, rather than synthetic pigments. Classes of natural pigments include anthocyanins, carotenoids and chlorophylls. Preferably, the natural pigment is a known nutrient with health providing properties such as lycopene or betacarotene. Preferably, the pigment is oil-soluble and known to have bioavailability limited by its dissolution in aqueous environments like the mouth or human gastrointestinal tract. Preferably, the pigment is light-sensitive but has good stability to heat and acidic pH. Preferably, it is derived from either fruit, vegetable or plant extracts. Examples of natural yellow pigments include curcumin and lutein. Chlorophyll is an example of a natural green pigment. Examples of natural red pigments include anthocyanin, carmine, betanin, and lycopene. Brown pigments include a range of caramel colorants. Examples of natural orange pigments include betacarotene, annatto and paprika. Examples of fruit and vegetable extracts include black carrot, blackcurrant, blueberry, elderberry, grape, red cabbage, hibiscus and beetroot.

The pigments may also be nature identical in that they are synthesized artificially, but are chemically and functionally comparable to their natural counterparts. Examples include betacarotene, apocarotenal, and canthaxanthin. The pigments may also be derived from natural microorganisms. An example is the red/blue phycobilliproteins from microalgae. Another is the blue spirulina pigment from two species of cyanobacteria.

Mixtures of any of the above active ingredients may also be used.

### Chewing Gum Compositions

The term chewing gum composition as used herein includes chewing gum and bubble gum. The general constituents of chewing gum and bubble gum are well known to the skilled person.

Essentially, chewing gum comprises a gum base and other optional additives such as sweetener, flavouring, softener, colour, comprised in a water-soluble phase. Gum bases for use in chewing gum are well known and are a typically non-nutritive, non-digestible, water-insoluble masticatory delivery system which are used to carry sweetener, flavour and any other desired substances in chewing gum. Gum base provides the basic textural and masticatory properties of gum. Bubble gum bases are formulated with the ability to blow bubbles and typically contain higher levels of elastomers or higher molecular weight polymers.

Gum base typically comprises one or more of the following: elastomers, resins, fats, emulsifiers, fillers, antioxidants. Elastomers provide a degree of elasticity and can be natural latexes, e.g. couma macrocarpa, loquat, tunu, jelutong or chicle or synthetic rubbers such as styrene-butadiene rubber, butyl rubber, polyisobutylene. Resins provide strength and may be selected from wax. Fats may provide a plasticizing function and may be derived from hydrogenated vegetable oils. Emulsifiers may assist in hydrating the gum base; typical examples are lecithin, glycerol monostearate. Fillers may be used to impart texture and typical examples include calcium carbonate, talc. Antioxidants protect from oxidation and thereby extend shelf-life. A suitable antioxidant includes butylated hydroxytoluene (BHT). Suitable softeners include glycerine or vegetable oil and are typically used to blend the other ingredients and help prevent the gum from becoming hard or stiff.

Typically, chewing gum contains 20-25 wt % gum base based on the total weight of the chewing gum and bubble gum typically contains about 15 to 20 wt % gum base.

The manufacturing process typically involves melting the gum base until it has the viscosity of a thick syrup which is then filtered through a fine mesh screen. It may be further refined by separating dissolved particles in a centrifuge and further filtered. The clear base gum whilst still hot and melted may be put into mixing vats and further ingredients combined, which include; sweetener, humectant, softener, food colour, flavouring, preservative and other optional additives. The loaded porous silicon may also be combined at this stage with the mixture. The homogenised mixture may then be poured onto cooling belts and cooled with cold air. The cooled mixture may then be extruded, rolled, cut and shaped. The pieces of gum are then allowed to set, typically for about 24 to 48 hours. If the gum is to be coated, then they undergo further operations. For example, the pieces of gum may be wrapped with an undercoating for improved binding with outer layers. The porous silicon, which may be loaded with one or more ingredients, may be combined with the gum composition at various stages of the production process. For example, it may be combined during the gum melting and/or mixing stage and/or when the gum is coated.

### EXAMPLES

The invention will now be described by way of example only and without limitation with reference to the following Examples.

### Example 1

### Fade Resistance

Mesoporous silicon membranes were fabricated by anodization of 0.01 ohm cm p-type silicon wafers in hydrofluoric acid based electrolyte. These membranes were then milled to provide microparticles of 70 vol % porosity possessing a d₉₀ of 40 μm. The microparticles were then subjected to thermal oxidation in air at 800° C. for varying periods of time to generate structures with silicon to oxygen atomic ratios in the range 1.8 to 1.99, as measured by Energy Dispersive X-Ray Analysis. This generated powders with off-white to pale brown colouration, depending on the precise silicon content. Liquid pigments were then slowly mixed into batches of the powder up to the 0.5 ml/g level which was below the “wet point” of the porous material. The free flowing pigmented powder had a hue that closely resembled that of the liquid pigment. Pigmented powder was then either incorporated into a surface layer of uncoloured gum sticks, or blended throughout the gum matrix. In both cases, a uniform colouration of the gum was achieved. The gum coloured by the pigmented porous silicon powders was then subjected to various light stability tests and compared with gum coloured by liquid pigment and also gum coloured by pigmented pure porous silica particles. Gum sticks had half their surfaces protected by aluminium foil, and the other half exposed to: intense blue light (1200 mW/cm² at 450 nm for 240 seconds at 20° C.), or long wave UV (7 mW/cm² at 325 nm for 20 hours at 40° C.), or shortwave UV (10 mins at 254 nm at 40° C.). The gum sticks coloured with either natural liquid pigment or natural pigmented silica showed visually obvious fading under UV irradiation. The gum sticks coloured with natural pigmented porous silicon powder showed slight changes in hue but dramatically improved fade resistance and good uniformity of colour.

### Example 2

### Mouthfeel and Particle Retention

A block of chewing gum was softened by microwave heating and then mesoporous silicon particles of 70 vol % porosity possessing a d₅₀ of 20 μm and a d₉₀ of 47 μm were mixed in at loading levels in the range of 0.05 to 0.3 wt %. The gum blocks were cut into 1 g sticks. A taste panel chewed the gum sticks containing mesoporous silicon and control gum sticks which did not contain any added silicon. The chewing was conducted blind for 3 minutes. None of the tasters reported any adverse texture or grittiness associated with the silicon containing gum sticks. Both the chewed gum and the saliva were examined after the chew-out testing and demonstrated excellent retention of the mesoporous silicon microparticles within the gum.

### Example 3

### Volatile Flavour Retention and Triggered Release

A mesoporous silicon sample was loaded with a volatile model flavour oil (ethyl butyrate) at ambient temperature and pressure at a payload of about 61 wt %. The loaded sample was spray dry coated with a gum Arabica and maltodextrin blend. FTIR analysis indicated flavour retention during the microencapsulation process. Subsequent water immersion after 1 week of storage in ambient air triggered the release of the characteristic pineapple aroma of the volatile flavour. The same flavoured particles were loaded into gum sticks and upon chewing the flavour was again detectable.

1. A chewing gum composition comprising porous silicon.

2. A chewing gum composition according to claim 1, wherein the porous silicon comprises mesoporous silicon and/or microporous silicon.

3. A chewing gum composition according to claim 2, wherein the porous silicon consists of or consists essentially of mesoporous silicon.

4. A chewing gum composition according to claim 2, wherein the porous silicon consists of or consists essentially of microporous silicon.

5. A chewing gum composition according to claim 1, wherein the porous silicon comprises microparticles and/or nanoparticles.

6. A chewing gum composition according to claim 1, wherein the porous silicon has been surface modified.

7. A chewing gum composition according to claim 6 wherein the surface modified porous silicon comprises or consists essentially of, or consists of, one or more of: derivatised porous silicon, partially oxidised porous silicon, porous silicon modified with silicon hydride surfaces, a capping layer.

8. A chewing gum composition according to claim 1, wherein the porous silicon is present in an amount of from 0.001 wt % to 10 wt % based on the total weight of the chewing gum composition.

9. A chewing gum composition according to claim 1, wherein the porous silicon comprises at least one ingredient for delivery to the human or animal teeth and/or other oral surfaces.

10. A chewing gum composition according to claim 9, wherein the other oral surfaces include one or more of the cheeks, tongue, throat, gums.

11. A chewing gum composition according to claim 9, wherein the ingredient is selected from one or more of the following: sweetener, flavour, breath freshening agent, vitamin, antimicrobial, antibacterial, remineralizing agent, anti-plaque agent, anti-gingivitis agent, anti-calculus agent, tooth whitening agent, herbal extract, pain-relief agent, sensate, cooling agent, warming agent, colouring agent (e.g. pigment), stimulant, essential oil, slimming agent, cholesterol lowering agent, anti-smoking agent, nutrient.

12. A chewing gum composition according to claim 9, wherein the at least one ingredient is present in the range, in relation to the loaded porous silicon, of 1 to 90 wt %.

13. A chewing gum composition according to claim 12, wherein the at least one ingredient is present in the range, in relation to the loaded porous silicon, of 30 to 60 wt %.

14. A chewing gum composition according to claim 2 and wherein the mesoporous and/or microporous silicon has an oxygen to silicon atomic ratio of about 1.5 to 1.99.

15. A chewing gum composition according to claim 14, wherein the ratio is about 1.8 to 1.99.

16. A chewing gum composition according to claim 14, wherein the mesoporous and/or microporous silicon is loaded with a colouring agent.

17. A production process for preparing the chewing gum composition according to claim 1 comprising blending the porous silicon and other components of the chewing gum composition.

18. A method of treating and/or cleaning the teeth of a human or animal comprising chewing a chewing gum composition as claimed claim 1.

19. A method according to claim 18, wherein the method is a cosmetic method.

20. The use of porous silicon in a chewing gum composition as claimed in claim 1, for maintaining the colour or slowing the degree of discolouration of the chewing gum composition.
